(19) United States
(12) Patent Application Publication
(10) Pub. No.: US 2025/0051518 A1
(43) Pub. Date: Feb. 13, 2025

(54) METHOD FOR PRODUCING POLYHYDROXYALKANOATE AND USE OF SAME

(71) Applicant: KANEKA CORPORATION, Osaka-shi (JP)

(72) Inventors: Naoki DEGUCHI, Takasago-shi (JP); Masaru HIRANO, Takasago-shi (JP)

(73) Assignee: KANEKA CORPORATION, Osaka-shi (JP)

(21) Appl. No.: 18/718,979

(22) PCT Filed: Dec. 7, 2022
(86) PCT No.: PCT/JP2022/045100

§ 371 Date: Jun. 12, 2024

(30) Foreign Application Priority Data
Dec. 23, 2021 (JP) 2021-209798

(51) Int. Cl. C08G 63/90; C08G 63/06; C08G 63/89; C12P 7/625

(52) U.S. Cl. CPC C08G 63/90; C08G 63/06; C08G 63/89; C12P 7/625

(57) ABSTRACT

An object is to provide a PHA production method which enables efficient filtration. The above problem is solved by providing a PHA production method including a filtration step of subjecting, to dead-end filtration, an aqueous PHA suspension having a pH of 2.5 to 5.5 with use of a filter medium having an air permeability of 0.01 cc/cm²/sec to 5.0 cc/cm²/sec, the aqueous PHA suspension containing PHA surface adhesion protein in an amount of not more than 2,000 ppm, the aqueous PHA suspension having a liquid density of 0.50 g/mL to 1.08 g/mL in the filtration step.

### TECHNICAL FIELD

The present invention relates to a method for producing a polyhydroxyalkanoate and use of the polyhydroxyalkanoate.

### BACKGROUND ART

A polyhydroxyalkanoate (hereinafter, also referred to as a “PHA”) is known to be biodegradable.

A PHA produced by microorganisms is accumulated in the microbial cells of the microorganisms. Accordingly, in order to use a PHA as plastic, it is necessary to carry out a step of isolating a PHA from the microbial cells of the microorganisms and purifying the PHA. In the step of isolating and purifying a PHA, the microbial cells of PHA-containing microorganisms are disrupted or the biological components, excluding the PHA, are solubilized, before from an aqueous suspension thus obtained, the PHA is extracted. In this extraction, isolation operations such as, for example, centrifugation, filtration, drying are carried out.

As the PHA production method in which filtration is used, disclosed in, for example, Patent Literature 1 is a PHA production method including: a step of inoculating a fermentation medium with a species of PHA fermenter to cause fermentation; a step of subjecting a fermentation liquid to solid-liquid separation to obtain a fermentation supernatant liquid and a microbial cell precipitate; and a step of precipitating microbial cells and disrupting the cell walls of the microbial cells, and subjecting the disrupted cell walls to plate-and-frame filtration with use of a precoated filter, to obtain the PHA.

In addition, Patent Literature 2 discloses a method for recovering a PHA from the product of culturing cells and purifying the PHA, the method including a step of carrying out an acid treatment, which is a pretreatment.

### CITATION LIST

### Patent Literature

### [Patent Literature 1]

Specification of Chinese patent No. 111500650

### [Patent Literature 2]

International Publication No. WO 2015/015395

### SUMMARY OF INVENTION

### Technical Problem

However, the techniques described above are susceptible of further improvement from the viewpoint of a filtrate permeability and a leakage ratio.

An object of an aspect of the present invention is to provide a PHA production method which enables efficient filtration.

### Solution to Problem

The inventors of the present invention diligently conducted a study to solve the above problem. As a result the inventors obtained a novel finding which indicates that by controlling, in a filtration step of PHA production, the pH of an aqueous PHA suspension, the air permeability of a filter medium, the amount of PHA surface adhesion protein, and the liquid density of the aqueous PHA suspension, it is possible to achieve a specific filtrate permeability and a specific leakage ratio (i.e., it is possible to carry out efficient filtration). This led to the completion of the present invention.

Therefore, an aspect of the present invention is a PHA production method (hereinafter, referred to as the “present production method”) which includes a filtration step of subjecting, to dead-end filtration, an aqueous PHA suspension having a pH of 2.5 to 5.5 with use of a filter medium having an air permeability of 0.01 cc/cm²/sec to 5.0 cc/cm²/sec, the aqueous PHA suspension containing PHA surface adhesion protein in an amount of not more than 2,000 ppm, the aqueous PHA suspension having a liquid density of 0.50 g/mL to 1.08 g/mL in the filtration step.

Further, an aspect of the present invention is a PHA agglutinate (hereinafter, referred to as the “present PHA agglutinate”) having a Feret diameter of 1 mm to 100 mm and having a moisture content of 25.0% to 50.0% (W.B.).

### Advantageous Effects of Invention

With an aspect of the present invention, it is possible to provide a PHA production method which enables efficient filtration.

### BRIEF DESCRIPTION OF DRAWINGS

FIG. 1 illustrates an example measurement of the Feret diameter of a PHA agglutinate in accordance with Example 10 of the present invention.

### DESCRIPTION OF EMBODIMENTS

The following description will discuss an embodiment of the present invention in detail. The expression “A to B” representing a numerical range means “not less than A and not more than B” unless otherwise specified herein. All of the documents cited herein are incorporated herein by reference.

### 1. Outline of Present Invention

There was a problem of the difficulty in filtering a PHA produced within microbial cells due to the particle size of the PHA of approximately 1 μm to 2 μm. Further, since the PHA is recovered mainly by centrifugation, and the form of isolation is an aqueous suspension liquid, the PHA is recovered with a large amount of water contained. It is therefore necessary to evaporate the water contained in the aqueous suspension to separate the PHA from the water and recover the PHA. This presents a problem such as the need for a large amount of energy.

As a PHA production method in which filtration is used, the methods disclosed in, for example, Patent Literatures 1 and 2 described above are known. With the technique disclosed in Patent Literature 1, in which filtration is carried out before purification (in a state where many biological residues remain), there is the problem of an extremely low filtration rate due to the presence of impurities. Further, the inventors of the present invention carried out the replication of the technique disclosed in Patent Literature 2, and found that the pH ranged from 6 to 7. A problem found from this finding is that when filtration is carried out under this condition, a considerably large amount of resin would leak. Furthermore, the inventors found that when the mesh of a filter cloth is finer under the same condition, there is a problem of extremely low filtration rate.

To address this, the inventors of the present invention conducted a diligent study of the filtration step of PHA production from the viewpoint of a filtrate permeability and a leakage ratio. As a result, the inventors became the first to find that by controlling the pH of an aqueous PHA suspension, the air permeability of a filter medium, the amount of PHA surface adhesion protein, and the liquid density of the aqueous PHA suspension, it is possible to achieve a specific filtrate permeability and a specific leakage ratio (e.g., it is possible to carry out efficient filtration).

With the present production method, it is possible to provide a PHA production method which enables efficient filtration. The present production method therefore has a great advantage in industrial production of a PHA. As used herein, the phrase “enabling efficient filtration” means that the filtrate permeability is not less than 200 L/m²/hr and the leakage ratio is not more than 5%.

In addition, with the configuration as described above, it is possible to reduce the quantity of heat, and time and costs, i.e., energy, required in the drying step carried out after the filtration. This enables contribution to achievement of sustainable development goals (SDGs) including, for example, Goal 7 “Ensure access to affordable, reliable, sustainable and modern energy for all”. Here is a detailed description of the present invention.

### [2. PHA Production Method]

The present production method includes the following step:
a step (hereinafter, referred to as a step (d)) of subjecting, to dead-end filtration, an aqueous PHA suspension having a pH of 2.5 to 5.5 with use of a filter medium having an air permeability of 0.01 cc/cm²/sec to 5.0 cc/cm²/sec, the aqueous PHA suspension containing PHA surface adhesion protein in an amount of not more than 2,000 ppm, the aqueous PHA suspension having a liquid density of 0.50 g/mL to 1.08 g/mL in the filtration step.

Further, in an embodiment of the present invention, the present production method preferably includes, in addition to the above step (d), at least one of the following steps.
Step (a): a step (also referred to as a “solubilization step”) of disrupting and solubilizing cell-derived components of a microbial cell which contains a PHA, the cell-derived components excluding the PHA, the PHA having a volume median diameter of 0.5 μm to 5.0 μm
Step (b): a step (also referred to as a “recovery step”) of recovering an aqueous PHA suspension by centrifugation, after the step (a)
Step (c′): a step (also referred to as an “adjustment step”) of adjusting the pH of the aqueous PHA suspension obtained in the step (b) to 2.5 to 5.5
Step (c): a step (also referred to as a “heat treatment step”) of subjecting the aqueous PHA suspension to a heat treatment so that the aqueous PHA suspension has a temperature of 60° C. to 120° C.
Step (e): a step (also referred to as a “drying step”) of drying the PHA obtained in step (d) at 20° C. to 160° C.
Step (f): a step (also referred to as a “redispersion step”) of redispersing, in an aqueous solvent, the PHA dried to obtain an aqueous PHA suspension which contains a PHA having a volume median diameter of 0.5 μm to 5.0 μm

In the present production method, the above steps are preferably carried out in the order of the steps (a), (b), (c′), (c), (d), (e), and (f). However, the order can be changed as appropriate, according to purposes. For example, the order of the steps (a) and (b) can be exchanged so that the steps are carried out in the order of the step (b) and then the step (a), and the order of the steps (c′) and (c) can be exchanged so that the steps are carried out in the order of the step (c) and then the step (c′). Further, according to purposes, each of the steps (a), (b), (c′), and (c) can be carried out twice or more. This means that an order which is, for example, the order of the step (b), the step (a), and then the step (b) or the order of the step (c′), the step (c), and then the step (c′) is possible. The steps (a), (b), (c′), (c), (d), (e), and (f) will be described below in this order for convenience. In this specification, an aqueous suspension containing at least a PHA can be expressed as an “aqueous PHA suspension” for short.

### (Step (a))

In the step (a) of the present production method, cell-derived components of a microbial cell which contains a PHA, the cell-derived components excluding the PHA, are disrupted and solubilized. By disrupting and removing impurities (cell walls, protein, etc.) derived from the microbial cells through the step (a), a PHA having a volume median diameter of 0.5 μm to 5.0 μm can be efficiently recovered from the microbial cells.

### <PHA>

As used herein, the term “PHA” is a generic term for polymers the monomer unit of which is a hydroxyalkanoic acid. The hydroxyalkanoic acid of the PHA is not particularly limited, but examples thereof include 3-hydroxybutanoic acid, 4-hydroxybutanoic acid, 3-hydroxypropionic acid, 3-hydroxypentanoic acid, 3-hydroxyhexanoic acid, 3-hydroxyheptanoic acid, and 3-hydroxyoctanoic acid. The polymer the monomer unit of which is a hydroxyalkanoic acid may be a homopolymer, or may be a copolymer which contains two or more kinds of monomer units.

More specifically, examples of the PHA include poly(3-hydroxybutyrate) (P3HB), poly(3-hydroxybutyrate-co-3-hydroxyhexanoate) (P3HB3HH), poly(3-hydroxybutyrate-co-3-hydroxyvalerate) (P3HB3HV), poly(3-hydroxybutyrate-co-4-hydroxybutyrate) (P3HB4HB), poly(3-hydroxybutyrate-co-3-hydroxyoctanoate) (P3HB3HO), poly(3-hydroxybutyrate-co-3-hydroxyoctadecanoate) (P3HB3HOD), poly(3-hydroxybutyrate-co-3-hydroxydecanoate) (P3HB3HD), and poly(3-hydroxybutyrate-co-3-hydroxyvalerate-co-3-hydroxyhexanoate) (P3HB3HV3HH). In particular, the PHA is preferably P3HB, P3HB3HH, P3HB3HV, or P3HB4HB, which is industrially easily produced.

Further, the PHA is more preferably P3HB3HH, which is a copolymer of 3-hydroxybutyric acid and 3-hydroxyhexanoic acid, from the following viewpoints: changing the composition ratio of repeating units makes it possible to change the melting point and crystallinity and thereby change the physical properties, such as Young's modulus and heat resistance, and impart a physical property intermediate between those of polypropylene and polyethylene; and P3HB3HH is easy to industrially produce as described above, and is a useful plastic in terms of physical properties.

According to an embodiment of the present invention, the composition ratio of repeating units of P3HB3HH is such that the composition ratio of a 3-hydroxybutyrate unit to a 3-hydroxyhexanoate unit is preferably 80/20 (mol/mol) to 99.9/0.1 (mol/mol) and more preferably 85/15 (mol/mol) to 97/3 (mol/mol), from the viewpoint of the balance between plasticity and strength. When the composition ratio of a 3-hydroxybutyrate unit to a 3-hydroxyhexanoate unit is not more than 99.9/0.01 (mol/mol), a sufficient plasticity is obtained, and when the composition ratio is not less than 80/20 (mol/mol), a sufficient hardness is obtained.

In the step (a), the PHA has a volume median diameter which is preferably not more than 50 times, more preferably not more than 20 times, and even more preferably not more than 10 times the volume median diameter (hereinafter referred to as a “primary particle size”) of primary particles of the PHA. When the volume median diameter of the PHA is not more than 50 times the primary particle size, the aqueous PHA suspension exhibits more excellent flowability, and the productivity of the PHA further improves accordingly.

According to an embodiment of the present invention, the volume median diameter of the PHA is, for example, preferably 0.5 μm to 5.0 μm, more preferably 1.0 μm to 4.5 μm, and even more preferably 1.0 μm to 4.0 μm, from the viewpoint of achieving excellent flowability. The volume median diameter of the PHA is measured with use of a laser diffraction/scatter particle size distribution meter LA-950 manufactured by HORIBA.

### <Microbial Cell (Microorganism)>

Microorganisms used in the step (a) are not particularly limited provided that the microorganisms can produce a PHA in the cells thereof. The microorganisms that can be used are, for example, a microorganism isolated from nature, a microorganism deposited with a depositary institution (e.g., IFO and ATCC) for microbial strains, or mutants and transformants that can be prepared from the aforementioned microorganisms. Examples of a microbial cell that produces P3HB, which is an example PHA, include Bacillus megaterium, which is the first P3HB-producing microbial cell that discovered in 1925, and also include other natural microorganisms such as Cupriavidus necator (former classification: Alcaligenes eutrophus, Ralstonia eutropha) and Alcaligenes latus. These microorganisms are known to have a PHA accumulated within the cells thereof.

Examples of a microbial cell that produces a copolymer of hydroxybutyrate and another hydroxyalkanoate, the copolymer being an example of the PHA, include: Aeromonas caviae, which is a P3HB3HV and P3HB3HH-producing microorganism; and Alcaligenes eutrophus, which is a P3HB4HB-producing microorganism. In particular, regarding P3HB3HH, the microbial cell is more preferably Alcaligenes eutrophus AC32 strain (FERM BP-6038), which has introduced therein genes of a group of PHA synthetases, (T. Fukui, Y. Doi, J. Bacteriol., 179, pp. 4821 to 4830 (1997)), or the like, for the purpose of enhancing the productivity of P3HB3HH. Besides the above, the microbial cell may be of a genetically engineered microorganism which has introduced therein various types of PHA synthesis-related genes selected according to a desired PHA to be produced.

### <Disruption and Solubilization of Cell-Derived Components>

A method for disrupting and solubilizing cell-derived components of a microbial cell which contains a PHA, the cell-derived components excluding the PHA, in the step (a) is not particularly limited.

According to an embodiment of the present invention, the above disruption and solubilization are carried out with use of, for example, a lytic enzyme and/or a proteolytic enzyme (e.g., alkaline proteolytic enzyme).

As used herein, the term “lytic enzyme” means an enzyme that has the activity of degrading the cell wall (e.g., peptidoglycan) of a microbial cell (the activity of dissolving a microbial cell).

According to an embodiment of the present invention, the lytic enzyme is not particularly limited, but examples thereof include lysozyme, labiase, β-N-acetylglucosaminidase, an endolysin, and an autolysin. The lytic enzyme is preferably lysozyme from the viewpoint of economic advantage. One of these lytic enzymes may be used alone, or two or more of these lytic enzymes may be used in combination.

As the lytic enzyme, a commercially available product can be used, and examples thereof include “Lysozyme” and “Achromopeptidase” manufactured by FUJIFILM Wako Pure Chemical Corporation.

According to an embodiment of the present invention, the optimum pH of the lytic enzyme is not particularly limited, provided that the lytic enzyme has cell wall degradation activity, but is, for example, 5.0 to 11.0, preferably 6.0 to 9.0, and more preferably 6.0 to 8.0.

According to an embodiment of the present invention, the optimum temperature of the lytic enzyme is not particularly limited, but is preferably not higher than 60° C. and more preferably not higher than 50° C., from the viewpoint of eliminating the need for undue heating to enable prevention of a thermal change (pyrolysis) of a PHA. The lower limit of the optimum temperature is not particularly limited, but is preferably not lower than room temperature (e.g., 25° C.), from the viewpoint of eliminating the need for an undue cooling operation and therefore being economical.

As used herein, the term “alkaline proteolytic enzyme” means a proteolytic enzyme having the activity of degrading protein in an alkaline environment (e.g., in a solution having a pH of 8.5).

According to an embodiment of the present invention, the alkaline proteolytic enzyme is not particularly limited, provided that the alkaline proteolytic enzyme has the activity of degrading protein in an alkaline environment, but examples thereof include serine-specific proteolytic enzymes (e.g., subtilisin, chymotrypsin, and trypsin), cysteine-specific proteolytic enzymes (e.g., papain, bromelain, and cathepsin), and aspartic acid-specific proteolytic enzymes (e.g., pepsin, cathepsin D, and HIV protease). The alkaline proteolytic enzyme is preferably a serine-specific proteolytic enzyme, and is, in particular, subtilisin (e.g., alcalase), from the viewpoint of economic advantage. One of these alkaline proteolytic enzymes may be used alone, or two or more of these alkaline proteolytic enzymes may be used in combination.

As the alkaline proteolytic enzyme, a commercially available product can be used, and examples thereof include: “Alcalase 2.5L” manufactured by Novozyme; “Protin SD-AY10” and “Protease P” Amano “3SD” manufactured by Amano Enzyme Inc.; “Multifect PR6L” and “Optimase PR89L” manufactured by Danisco Japan Ltd.; “Sumizyme MP” manufactured by Shin Nihon Chemical Co., Ltd.; “Delvolase” manufactured by DSM Japan K.K.; “Biopurase OP”, “Biopurase SP-20FG”, and “Biopurase SP-4FG” manufactured by Nagase ChemteX Corporation; “Orientase 22BF” manufactured by HBI Enzymes Inc.; and “AROASE XA-10” manufactured by Yakult Pharmaceutical Industry Co., Ltd.

According to an embodiment of the present invention, the optimum pH of the alkaline proteolytic enzyme is not particularly limited, provided that the alkaline proteolytic enzyme has activity in an alkaline environment, but is, for example, 8.0 to 14.0, preferably 8.0 to 12.0, more preferably 8.0 to 10.0, even more preferably 8.0 to 9.0, and most preferably 8.5.

According to an embodiment of the present invention, the optimum temperature of the alkaline proteolytic enzyme is not particularly limited, but is preferably not higher than 60° C., and more preferably not higher than 50° C., from the viewpoint of eliminating the need for undue heating to enable prevention of a thermal change (pyrolysis) of a PHA. The lower limit of the optimum temperature is not particularly limited, but is preferably not lower than room temperature (e.g., 25° C.), from the viewpoint of eliminating the need for an undue cooling operation and therefore being economical.

According to an embodiment of the present invention, the disruption and solubilization of the cell-derived components in the step (a) can be carried out with use of lysozyme and alcalase in combination.

A time during which the above enzyme treatment is carried out in the step (a) can vary according to the type, pH, temperature, etc. of an enzyme, but is, for example, one hour to eight hours, and preferably two hours to six hours.

A solvent (the “solvent” may also be referred to as an “aqueous medium”) of the aqueous PHA suspension in the present production method may be water or a mixed solvent of water and an organic solvent. The mixed solvent contains an organic solvent compatible with water in a concentration which is not particularly limited, provided that the concentration is not higher than the aqueous solubility of the organic solvent to be used. The organic solvent compatible with water is not particularly limited, but examples thereof include: alcohols such as methanol, ethanol, 1-propanol, 2-propanol, 1-butanol, 2-butanol, iso-butanol, pentanol, hexanol, and heptanol; ketones such as acetone and methyl ethyl ketone; ethers such as tetrahydrofuran and dioxane; nitriles such as acetonitrile and propionitrile; amides such as dimethylformamide and acetamide; dimethyl sulfoxide; pyridine; and piperidine. In particular, the organic solvent compatible with water is preferably methanol, ethanol, 1-propanol, 2-propanol, 1-butanol, 2-butanol, iso-butanol, acetone, methyl ethyl ketone, tetrahydrofuran, dioxane, acetonitrile, propionitrile, or the like, in terms of being easily removed. Further, the organic solvent compatible with water is more preferably methanol, ethanol, 1-propanol, 2-propanol, butanol, acetone, or the like in terms of being easily available. Furthermore, the organic solvent compatible with water is particularly preferably methanol, ethanol, and acetone. Note that the aqueous medium of the aqueous PHA suspension is allowed to contain another solvent, a component derived from a microbial cell, a compound produced during purification, and the like, provided that the spirit of the present invention is not impaired.

The aqueous medium of the aqueous PHA suspension in the present production method preferably contains water. The water content of the aqueous medium is preferably not less than 5% by weight, more preferably not less than 10% by weight, even more preferably not less than 30% by weight, and particularly preferably not less than 50% by weight.

### (Other Steps)

According to an embodiment of the present invention, the present production method may include the following steps before the step (a).

<Step (a1)>

A step (a1) is a step of culturing a microbial cell which contains a PHA.

A microbial cell used in the step (a1) is, for example, a microbial cell described in the section <Microbial cell (microorganism)> above.

In the step (a1), a method for culturing the microbial cell is not particularly limited, but examples thereof include the method described in paragraphs [0041] to [0048] of the International Publication No. WO 2019/142717.

<Step (a2)>

A step (a2) is a step of inactivating microbial cells obtained in the step (a1). In this step, the microbial cells obtained in the step (a1) are inactivated, and an inactivated culture solution is obtained.

In the step (a2), a method for the inactivation is not particularly limited, but examples thereof include a method of heating and stirring the culture solution which contains P3HA-containing microbial cells at the temperature of the culture solution of 60° C. to 70° C. for seven hours.

<Step (a3)>

A step (a3) is a step of adjusting the concentration and pH of the inactivated culture solution obtained in the step (a2). The step (a3) is mainly carried out in a case where the viscosity of the inactivated culture solution obtained in the step (a2) is high, and the concentration and pH of the inactivated culture solution are adjusted so that the viscosity of the inactivated culture solution is reduced. Carrying out the step (a3) facilitates the solubilization in the step (a).

A method for adjusting the concentration and pH of an inactivated culture solution in the step (a3) is not particularly limited, but is carried out by any method used in the art. For example, by the addition of hydrogen peroxide, etc. to the inactivated culture solution, the concentration of the inactivated culture solution can be adjusted. Further, examples of a method for adjusting the pH include a method of adding a basic compound to the inactivated culture solution. The basic compound is not particularly limited, but is preferably an alkali metal hydroxide or an alkaline-earth metal hydroxide, and more preferably sodium hydroxide. One of the basic compounds may be used alone, or two or more of the basic compounds may be used in combination.

### (Step (b))

In the step (b) of the present production method, an aqueous PHA suspension is recovered by centrifugation after the step (a). With the step (b), it is possible to remove impurities (cell walls, protein, etc.) derived from the microbial cells contained in the aqueous PHA suspension.

In the step (b), the recovery of the aqueous PHA suspension is carried out by any centrifugation method known in the art. The method of centrifugation is not particularly limited, but examples thereof include centrifugation with use of a sedimentation centrifuge, a centrifugal dewaterer, or the like.

Examples of the sedimentation centrifuge include sedimentation centrifuges of a separator type (such as disc type, self-cleaning type, nozzle type, screw decanter type, or skimming type), a cylindrical type, and a decanter type. According to a sediment component discharging method, each of the sedimentation centrifuges is categorized into a batch-wise or continuous sedimentation centrifuge. Similarly, the centrifugal dewaterer is categorized into a batch-wise or continuous centrifugal dewaterer. By using these devices, it is possible to separate a sediment containing a PHA from a culture solution component with use of a specific gravity difference.

Since the amount of impurities which are to remain in an end product depends mainly on the steps (a) and (b), it is preferable to reduce the impurities to the smallest possible amount. As a matter of course, in some uses, the impurities are allowed to be mixed to the extent that the physical properties of the end product are not impaired. However, in a case where a highly pure PHA is required, such as the case of a medical use, it is preferable to reduce the impurities to the smallest possible amount. An index of the degree of purification in reducing the impurities can be, for example, the amount of PHA surface adhesion protein contained in the aqueous PHA suspension. The amount of such protein with respect to the weight of the PHA is not more than 2,000 ppm, preferably not more than 1,900 ppm, even more preferably not more than 1,800 ppm, and most preferably not more than 1,700 ppm. The amount of PHA surface adhesion protein in the aqueous PHA suspension falling within the above range provides the advantage that the leakage ratio is not too high. An inferred reason why this effect is brought about is that when the amount of PHA surface adhesion protein is small, PHAs easily gather.

(Step (c′))

In the step (c′), the aqueous PHA suspension recovered by centrifugation usually has a pH greater than 7. In the step (c′) of the present production method, the pH of the aqueous PHA suspension obtained in the step (b) is adjusted to 2.5 to 5.5. Carrying out the pH adjustment of the step (c′) reduces the leakage ratio in the filtration of the step (d).

In the step (c′), the pH of the aqueous PHA suspension is 2.5 to 5.5, preferably 2.5 to 5.0, more preferably 2.5 to 4.5, even more preferably 2.5 to 4.0, and particularly preferably 2.5 to 3.5. The pH of the aqueous PHA suspension falling within the above range provides the advantage of being able to improve the filtrate permeability without enhancing the ratio in which a PHA leaks into the filtrate in the filtration step. An inferred reason why this effect is brought about is that PHAs are not too small, and thus easily agglutinate.

Further, the upper limit of the pH is preferably not more than 5.5 from the viewpoint of reducing the coloration during heating and melting of a PHA and ensuring the stability of the molecular weight during heating and/or drying, and also from the viewpoint of obtaining a PHA the coloration of which during heating and melting has been reduced and the molecular weight reduction of which during heating and/or drying has been prevented. The lower limit of the pH is preferably not more than 2.5 from the viewpoint of the resistance of a container to acid.

In the step (c′), a method for adjusting the pH is not particularly limited, but examples thereof include a method of adding acid. The acid is not particularly limited, but may be either an organic acid or an inorganic acid, and may or may not be volatile. More specifically, examples of the acid can include sulfuric acid, hydrochloric acid, phosphoric acid, and acetic acid.

According to an embodiment of the present invention, it is preferable that an additional pH adjustment should not be carried out after the pH adjustment in the step (c) and until after the step (d) is carried out.

### (Step (c))

In the step (c) of the present production method, the aqueous PHA suspension undergoes a heat treatment so as to have a temperature of 60° C. to 120° C. With the step (c), it is possible to increase the filtrate permeability during filtration.

In the step (c), the heat treatment is carried out so that the aqueous PHA suspension has a temperature which is preferably 60° C. to 120° C., more preferably 62° C. to 118° C., and even more preferably 65° C. to 115° C. When the temperature of the aqueous PHA suspension falls within the above range, it is possible to increase the filtrate permeability during filtration to a greater degree.

A method of the heat treatment in the step (c) is not particularly limited, but examples thereof include (i) a method of using steam to warm a container holding the aqueous PHA suspension, (ii) a method of using oil to warm a container holding the aqueous PHA suspension, and (iii) a method of directly putting steam into the aqueous PHA suspension. The temperatures of the steam of (i) and (iii) and the temperature of the oil of (ii) are not particularly limited, provided that the temperatures cause the aqueous PHA suspension in the step (c) to have a temperature of 60° C. to 120° C., and are, for example, 95° C. to 150° C.

### (Step (d))

In the step (d) of the present production method, the aqueous PHA suspension having a pH of 2.5 to 5.5 is subjected to dead-end filtration with use of a filter medium having an air permeability of 0.01 cc/cm²/sec to 5.0 cc/cm²/sec. In the step (d), the amount of PHA surface adhesion protein in the aqueous PHA suspension is not more than 2,000 ppm, and the liquid density of the aqueous PHA suspension in the filtration step is 0.50 g/mL to 1.08 g/mL. With the step (d), PHAs having a constant Feret diameter and a constant moisture content are obtained.

In this specification, the amount (cc) of air passing through a unit area (cm²) of a filter medium per second is referred to as an air permeability. In the step (d), the air permeability is 0.01 cc/cm²/sec to 5.0 cc/cm²/sec, preferably 0.1 cc/cm²/sec to 4.0 cc/cm²/sec, more preferably 0.2 cc/cm²/sec to 3.5 cc/cm²/sec, even more preferably 0.3 cc/cm²/sec to 3.0 cc/cm²/sec, and particularly preferably 0.4 cc/cm²/sec to 2.5 cc/cm²/sec. The air permeability falling within the above range provides the advantage of a low ratio in which a PHA leaks into the filtrate. The air permeability in the filtration step of the present production method is measured by the method described in Examples.

In the step (d), the liquid density of the aqueous PHA suspension is 0.50 g/mL to 1.08 g/mL, preferably 0.55 g/mL to 1.08 g/mL, more preferably 0.60 g/mL to 1.05 g/mL, and even more preferably 0.65 g/mL to 1.03 g/mL. The liquid density of the aqueous PHA suspension falling within the above range provides the advantage of a high filtrate permeability and a low moisture content in the PHA agglutinate. An inferred reason why the filtrate permeability decreases when the liquid density is low is that the aqueous PHA suspension has an increased viscosity through inclusion of air, and the air and a PHA interact with each other so that the viscosity is increased. The liquid density of the aqueous PHA suspension can be adjusted by, for example, inclusion of air. When the amount of the air is increased, the liquid density of the aqueous PHA suspension decreases. When the amount of the air is reduced, the liquid density of the aqueous PHA suspension increases.

The filter medium used in the step (d) is not particularly limited, but can be selected from among various materials such as, for example, paper, filter cloth (woven cloth, non-woven cloth), screens, sintered plates, porcelain filters, polymer membranes, punching metal, and wedge wires. A filter cloth is preferably used from the viewpoint of price and ease of cleaning.

A filtration method in the step (d) only needs to be dead-end filtration, and is not particularly limited. Examples of the filtration method include suction filtration, pressure filtration, centrifugal filtration, and gravitational filtration. In particular, suction filtration, pressure filtration, and centrifugal filtration are preferably used from the viewpoint of the size of the device. Furthermore, due to the simplicity of the structure, suction filtration and pressure filtration are more preferably used.

As used herein, the filtrate permeability means the rate at which a filtrate passes through a PHA agglutinate and a filter medium. In the step (d) of the present production method, the filtrate permeability is preferably not less than 200 L/m²/hr, more preferably not less than 300 L/m²/hr, more preferably not less than 400 L/m²/hr, more preferably not less than 500 L/m²/hr, more preferably not less than 600 L/m²/hr, even more preferably not less than 800 L/m²/hr, and particularly preferably not less than 1000 L/m²/hr. The filtrate permeability falling within the above range provides the advantage of making it possible to reduce operation time. The filtrate permeability being higher is more preferable. The upper limit of the filtrate permeability is not particularly limited, but is, for example, not higher than 4,000 L/m²/hr. The filtrate permeability is measured by the method described in Examples.

As used herein, the leakage ratio means a ratio in which a PHA leaks to a filtrate after the filtration step. In the step (d) of the present production method, the leakage ratio is preferably not more than 5%, more preferably not more than 4%, more preferably not more than 3%, more preferably not more than 2%, more preferably not more than 1%, even more preferably not more than 0.5%, and particularly preferably not more than 0.3%. The leakage ratio falling within the above range has the advantage of making it possible to separate a PHA from water with a high quality of separation, and thereby makes it possible to recover a PHA with a high yield. The leakage ratio is measured by the method described in Examples.

The temperature (filtration temperature) of the aqueous PHA suspension in the step (d) is preferably 20° C. to 95° C., more preferably 20° C. to 90° C., even more preferably 20° C. to 85° C., and particularly preferably 25° C. to 85° C. The temperature of the aqueous PHA suspension falling within the above range provides the advantage of a high filtrate permeability. The inferred reason for the increase in the filtrate permeability is that with an increase in temperature, the viscosity increases, and at the same time, the particle size increases.

In the present production method, in a case where the heat treatment step of the step (c) is included, the temperature of the aqueous PHA suspension during filtration is preferably not less than 5° C. lower, more preferably not less than 8° C. lower, even more preferably not less than 10° C. lower, and particularly preferably not less than 12° C. lower, than the temperature of the aqueous PHA suspension after the heat treatment step. The temperature of the aqueous PHA suspension during filtration falling within the above range provides the advantage of enabling filtration of a PHA at a high filtrate permeability. Further, a method for reducing the temperature after the heat treatment step is not particularly limited, but examples thereof include: cooling via a cooling device; and allowing the aqueous PHA suspension to cool.

In the step (d), the description of the “pH” made in (Step (c′)) above is employed. In addition, in the step (d), the description of the “amount of PHA surface adhesion protein contained in the aqueous PHA suspension” made in (Step (b)) above is also employed.

### (Step (e))

In the step (e) of the present production method, a PHA obtained in the step (d) is dried at 20° C. to 80° C. With the step (e), it is possible to evaporate water contained in the aqueous PHA suspension to adjust the moisture content.

A method for drying a PHA in the step (e) is not particularly limited, but examples thereof include: heating; vacuum drying; and drying at normal temperature. The method is preferably carried out by heating from the viewpoint of a moderate drying speed. A heating medium (e.g., hot air or a jacket) during the drying is preferably at 20° C. to 160° C., more preferably at 40° C. to 160° C., even more preferably at 40° C. to 150° C., and particularly preferably at 50° C. to 150° C.

### (Step (f))

In the step (f) of the present production method, the dried PHA is redispersed in an aqueous solvent so that an aqueous PHA suspension containing a PHA which has a volume median diameter of 0.5 μm to 5.0 μm is obtained. By carrying out the step (f) after the step (e), an aqueous PHA suspension is obtained, the aqueous PHA suspension containing a PHA which has a particle size substantially the same as the original particle size (primary particle size).

In the step (f), a method of the redispersion is not particularly limited, but is carried out by any method used in the art.

In the step (f), the volume median diameter of the PHA is not particularly limited, provided that the volume median diameter is substantially the same as the volume median diameter of the PHA in the step (a), but is, for example, preferably 0.5 μm to 5.0 μm, more preferably 1.0 μm to 4.5 μm, and even more preferably 1.0 μm to 3.0 μm.

### (3. PHA Agglutinate)

The present PHA agglutinate has a Feret diameter of 1 mm to 100 mm and a moisture content of 25.0% to 50.0% (W.B.). A PHA agglutinate may also be referred to as “PHA cake”, “filtration cake” or “PHA filtration cake”.

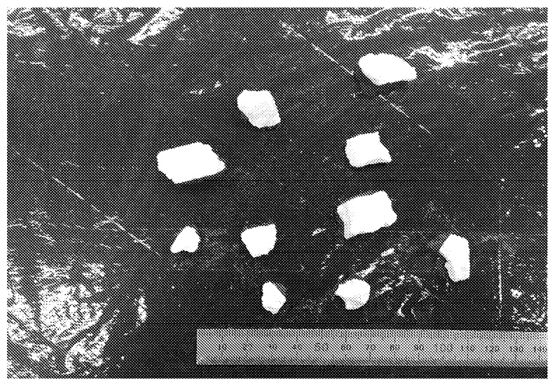

The Feret diameter of the present PHA agglutinate is 1 mm to 100 mm, preferably 1 mm to 70 mm, more preferably 1 mm to 50 mm, even more preferably 1 mm to 30 mm, and particularly preferably 1 mm to 10 mm. The Feret diameter of the present PHA agglutinate falling within the above range provides the advantage of advantageously transferring the present PHA agglutinate to the following process. It should be noted that it is possible to size the PHA agglutinate obtained in the step (d) such that the Feret diameter falls within the above range, by a mechanical method such as a crusher or a screw, or also by drop impact crushing. The Feret diameter of the present PHA agglutinate is determined by capturing an image (FIG. 1) of 10 PHA agglutinates, analyzing the image by ImageJ (ver1.50), finding the respective Feret diameters of the 10 PHA agglutinates, and taking the simple average of the respective 10 Feret diameters.

The moisture content of the present PHA agglutinate is 25.0% to 50.0% (W.B.), preferably 25.5% to 49.0% (W.B.), more preferably 26.0% to 48.0% (W.B.), even more preferably 26.5% to 47.0% (W.B.), and particularly preferably 27.0% to 46.0% (W.B.). The moisture content of the present PHA agglutinate falling within the above range brings the PHA agglutinate into a solid state, not into a slurry state, and thus provides the advantage of making the PHA agglutinate easy to put into a dryer. The moisture content of the present PHA agglutinate is measured by the method described in Examples.

According to an embodiment of the present invention, the present PHA agglutinate is produced by the present production method.

The present PHA agglutinate may contain various components which have been produced in the course of the present production method, or could not have been removed, provided that the present PHA agglutinate has the effect of the present invention.

The present PHA agglutinate can be used for various uses such as paper, a film, a sheet, a tube, a plate, a rod, a container (e.g. a bottle container), a bag, and a component.

The present invention is not limited to the embodiments, but can be altered by a skilled person in the art within the scope of the claims. The present invention also encompasses, in its technical scope, any embodiment derived by combining technical means disclosed in differing embodiments.

An aspect of the present invention includes the following.

<1> A polyhydroxyalkanoate production method including a filtration step of subjecting, to dead-end filtration, an aqueous polyhydroxyalkanoate suspension having a pH of 2.5 to 5.5 with use of a filter medium having an air permeability of 0.01 cc/cm²/sec to 5.0 cc/cm²/sec,
the aqueous polyhydroxyalkanoate suspension containing polyhydroxyalkanoate surface adhesion protein in an amount of not more than 2,000 ppm,
the aqueous polyhydroxyalkanoate suspension having a liquid density of 0.50 g/mL to 1.08 g/mL in the filtration step.
   <2> The polyhydroxyalkanoate production method described in <1>, in which in the filtration step, a filtrate permeability is not less than 200 L/m²/hr, and a leakage ratio is not more than 5%.
   <3> The polyhydroxyalkanoate production method described in <1> or <2>, in which the aqueous polyhydroxyalkanoate suspension has a temperature of 20° C. to 95° C. in the filtration step.
   <4> The polyhydroxyalkanoate production method described in any one of <1> to <3>, further including a step (a) of disrupting and solubilizing cell-derived components of a microbial cell containing a polyhydroxyalkanoate, the cell-derived components excluding the polyhydroxyalkanoate,
the polyhydroxyalkanoate having a volume median diameter of 0.5 μm to 5.0 μm in the step (a).
   <5> The polyhydroxyalkanoate production method described in <4>, further including a step (b) of recovering the aqueous polyhydroxyalkanoate suspension by centrifugation after the step (a).
   <6> The polyhydroxyalkanoate production method described in any one of <1> to <5>, further including a step of drying, at 20° C. to 80° C., a polyhydroxyalkanoate obtained in the filtration step.
   <7> The polyhydroxyalkanoate production method described in <6>, further including a step of redispersing, in an aqueous solvent, the polyhydroxyalkanoate dried, to obtain an aqueous polyhydroxyalkanoate suspension which contains a polyhydroxyalkanoate having a volume median diameter of 0.5 μm to 5.0 μm.
   <8> The polyhydroxyalkanoate production method described in any one of <1> to <7>, further including a heat treatment step of subjecting the aqueous polyhydroxyalkanoate suspension to a heat treatment before the filtration step so that the aqueous polyhydroxyalkanoate suspension has a temperature of 60° C. to 120° C.
   <9> The polyhydroxyalkanoate production method described in <8>, in which the aqueous polyhydroxyalkanoate suspension has not less than 5° C. lower temperature during filtration than after the heat treatment step.
   <10> The polyhydroxyalkanoate production method described in <5>, further including a step of adjusting a pH of the aqueous polyhydroxyalkanoate suspension obtained in the step (b) to 2.5 to 5.5.
   <11> A polyhydroxyalkanoate agglutinate having a Feret diameter of 1 mm to 100 mm and having a moisture content of 25.0% to 50.0% (W.B.).

### EXAMPLES

The following description will discuss the present invention in more detail on the basis of Examples. However, the present invention is not limited to Examples. In Examples, “P3HB3HH” is used as the “PHA”. The word “PHA” described in Examples can therefore be read as “P3HB3HH”.

### (Measurement Method)

Measurements in Examples and Comparative Examples were carried out by the following method.

### (Air Permeability)

The air permeability was measured by the method described in JIS L 1096. Specifically, with use of a Frazier type permeameter (Permeameter P2 manufactured by Toyo Seiki Seisaku-sho, Ltd.), the amount of air suction was adjusted such that an inclined manometer indicates 125 Pa, and an air flow rate was measured in such a situation.

### (Amount of PHA Surface Adhesion Protein)

The amount of PHA surface adhesion protein was measured with use of BCA Protein Assay Kit (manufactured by Thermo Fisher Scientific Inc.). Specifically, the aqueous PHA suspension immediately before the step (c′) was put in a 15-mL falcon tube in an amount of 20 mg to 50 mg (in this amount of aqueous PHA suspension, approximately 10 mg of P3HB3HH particles were contained), and 2 mL of a reagent of the kit was added. After that, the falcon tube was shaken at 60° C. for 30 minutes. Thirty minutes after the end of shaking, the aqueous PHA suspension was cooled to 25° C., and the absorbance at a wavelength of 562 nm was measured.

### (Solid Concentration)

The solid concentrations of the PHA-containing culture solution after the inactivation and the aqueous PHA suspension after the pH adjustment were measured with use of a heat drying type moisture analyzer ML-50 (manufactured by A&,D Company, Limited). The culture solution was heated at 130° C. until the rate at which the weight changed fell below 0.05%/min, and the solid concentration thereof was determined from the change in the weight before and after the heating.

### (Liquid Density)

Twenty mL of the aqueous PHA suspension immediately before the filtration step was heated to a filtration temperature, and then suctioned into a 20-mL plastic syringe (manufactured by Terumo Corporation) the weight of which was measured in advance. Next, the weight of the 20 mL of the aqueous suspension and the plastic syringe was measured, and a liquid density at the filtration temperature was calculated by dividing, by the volume (20 mL) of the liquid, a weight (g) obtained by subtracting the weight of the 20-mL plastic syringe from the measured weight.

### (Heat Treatment Temperature)

As the heat treatment temperature, the temperature of a part of the aqueous PHA suspension was measured, the part being the farthest from a heat source, the aqueous PHA suspension having been brought into a fluidized state with use of a stirring blade or the like. For example, in a case of heating a container from without, the center of the container was measured, and in a case of directly putting steam into the center of a container, the temperature of the wall of the container was measured. The temperature was measured with a type-K thermocouple (AD5601A manufactured by A&D Company, Limited).

### (Filtration Temperature)

As the temperature during filtration, the temperature immediately before the aqueous PHA suspension was put into a filter was measured with use of a type-K thermocouple (AD5601A manufactured by A&D Company, Limited).

(pH of Aqueous PHA Suspension in Step (c′))

A pH meter (9652-10D manufactured by HORIBA) was used in the measurement. The pH was measured at a position in the aqueous PHA suspension, the position being the farthest from a position where acid was added, the aqueous PHA suspension having been brought into a fluidized state with use of a stirring blade or the like. For example, in a case of adding acid at the wall of a container, the pH was measured at the center of the container.

### (Filtrate Permeability in Suction Filtration)

A filter cloth was placed on a filter (kst-47 manufactured by Advantec) having an inner diameter of 47 mm. The filter was then mounted in a suction bell (2L manufactured by SHIBATA) with the upper lid of the filter removed. A 50-mL graduated cylinder made of glass was placed in the suction bell so that the whole filtrate was accommodated in the graduated cylinder. The aqueous PHA suspension was put into the filter while suction was carried out via a vacuum pump such that the pressure is reduced to −76 kPa. The dropping filtrate was captured on a video camera. With use of a time (hr) required for the discharged amount of the filtrate to reach 25 mL after 5 mL of the filtrate was discharged, and the cross-sectional area (m²) of the filter, the filtrate permeability (L/m²/hr) was calculated by dividing the amount (L) of the difference (25−5=20 mL) in the filtrate by the time (hr) and the cross-sectional area (m²). The calculation was carried out with the cross-sectional area of the filter during the suction filtration in this specification regarded as the area of a circle having a diameter of 47 mm.

### (Filtrate Permeability in Pressure Filtration)

The aqueous PHA suspension in an amount of 2.0 kg was sent via a diaphragm pump so as to have a pressure of 0.2 MPa, and filtered via a press filter (YTO filter press manufactured by Yabuta Kikai Co. Ltd.) having a filtration area of 0.05 m². The filtrate permeability (L/m²/hr) was calculated from a time required for the discharged amount of the filtrate to reach 1,000 mL after 100 mL of the filtrate was discharged.

### (Leakage Ratio)

The weight of the filtrate obtained through the filtration step was measured. The absorbance of the filtrate was measured with use of a spectrophotometer (Jasco V-770 manufactured by JASCO Corporation), and the absorbance at a wavelength of 600 nm was measured. The PHA concentration in the filtrate was calculated with use of a calibration curve created from an aqueous PHA suspension having a known solid concentration. The leakage ratio (%) was calculated by finding the solid weight (g) of the filtrate from the PHA concentration of the filtrate and the weight (g) of the filtrate, and dividing the solid weight (g) of the filtrate by the solid weight (g) of the aqueous PHA suspension before the filtration step.

### (Moisture Content of PHA Agglutinate)

A PHA agglutinate obtained after the filtration was measured with use of a heat drying type moisture analyzer ML-50 (manufactured by A&,D Company, Limited). The PHA agglutinate was heated at 105° C. until the rate at which the weight changed fell below 0.05% (W.B.)/min, and the moisture content of the PHA agglutinate was determined from the change in the weight before and after the heating.

### (Volume Median Diameter)

The volume median diameter of a PHA was measured with use of a laser diffraction/scatter particle size distribution meter LA-950 manufactured by HORIBA.

### (Feret Diameter)

The Feret diameter of the present PHA agglutinate was determined by capturing an image (FIG. 1) of 10 PHA agglutinates, analyzing the image by ImageJ (ver1.50), finding the respective Feret diameters of the 10 PHA agglutinates, and taking the simple average of the respective 10 Feret diameters.

### Example 1

### (Preparation of Microbial Cell Culture Solution)

Ralstonia eutropha described in the International Publication No. WO 2019/142717 was cultured by a method described in paragraphs [0041] to [0048] of the same document, so that a microbial cell culture solution including microbial cells containing a PHA was obtained. Note that at present, Ralstonia eutropha is classified as Cupriavidus necator.

### (Inactivation)

The microbial cell culture solution obtained as described above was subjected to heating and stirring at the temperature of the microbial cell culture solution of 60° C. to 70° C. for seven hours for sterilization. An inactivated culture solution was thus obtained.

### (Viscosity Reduction Treatment)

To the inactivated culture solution obtained as described above, 35% by weight hydrogen peroxide (manufactured by FUJIFILM Wako Pure Chemical Corporation) was added, such that the hydrogen peroxide was 1% by weight of the inactivated culture solution. Next, a 30% aqueous sodium hydroxide was added so that the pH of the inactivated culture solution was adjusted to 11.0. By keeping on adding 30% aqueous sodium hydroxide while maintaining the solution at 60° C., the pH of the solution was maintained at 11.0 for 180 minutes. An aqueous PHA suspension was thus obtained.

### (Enzyme Treatment)

To the aqueous PHA suspension obtained as described above, 95% sulfuric acid was added, so that the pH of the aqueous PHA suspension was adjusted to 7.0±0.2. The solid concentration of the aqueous PHA suspension having sulfuric acid added thereto was measured, and found to be 30% by weight. After the addition of sulfuric acid, Lysozyme (manufactured by FUJIFILM Wako Pure Chemical Corporation), which is an enzyme that degrades sugar chains (peptidoglycan) in the cell walls, was added such that the concentration thereof in the aqueous PHA suspension was 10 ppm, and the aqueous PHA suspension was kept at 50° C. for two hours. After that, Alcalase 2.5L (manufactured by Novozyme), which is a proteolytic enzyme, was added such that the concentration thereof in the aqueous PHA suspension was 300 ppm, and 30% sodium hydroxide was then added at 50° C., so that the pH of the aqueous PHA suspension was adjusted to 8.5 and maintained for two hours.

### (Alkaline Treatment)

Sodium dodecyl sulfate (SDS, manufactured by Kao Corporation) was added to the above enzyme-treated solution so as to be 0.3% by weight of the enzyme-treated solution. After that, the pH of the enzyme-treated solution was adjusted to 11.0±0.2 with use of an aqueous sodium hydroxide solution. Next, the enzyme-treated solution was centrifuged (4,000 G, 10 minutes), and the supernatant was then removed. A twofold concentrated aqueous PHA suspension was thus obtained. The following operation was repeated 4 times: adding, to this concentrated aqueous PHA suspension, sodium hydroxide in an amount equal to that of the removed supernatant; centrifuging the aqueous PHA suspension again (4,000 G, 10 minutes); and removing a supernatant. The amount of PHA surface adhesion protein in the aqueous PHA suspension thus obtained was 1,000 ppm. The volume median diameter of the PHA was 2.2 μm.

### (Adjustment of pH)

The obtained aqueous PHA suspension was adjusted so as to have a solid concentration of 25% by weight, and kept at 60° C. Next, the pH was adjusted to 3.0 by the addition of 10% sulfuric acid. The liquid density was 1.00 g/mL.

### (Filtration)

The aqueous PHA suspension liquid was put in a water bath at 63° C., heated so as to have a temperature of 60° C., and subjected to suction filtration. When a filter cloth (T7104C manufactured by Yabuta Kikai Co. Ltd.) having an air permeability of 0.5 cc/cm²/sec was used, the filtrate permeability stood at 1,010 L/m²/hr, and the particle leakage ratio was 0.2%. A PHA agglutinate thus obtained had a moisture content of 40.3% (W.B.). The PHA agglutinate had a Feret diameter of 18 mm.

### Example 2

An aqueous PHA suspension having a pH of 3.0 was obtained by the same method as in Example 1, except that Alcalase 2.5L (manufactured by Novozyme), which is a proteolytic enzyme, was added in the enzyme treatment such that the concentration thereof in the aqueous PHA suspension was 200 ppm. The amount of PHA surface adhesion protein was 1,500 ppm, and the liquid density was 0.95 g/mL. The volume median diameter of the PHA was 2.3 μm. When filtration was carried out by the same method as in Example 1, the filtrate permeability stood at 580 L/m²/hr, and the leakage ratio was 1.0%. A PHA agglutinate thus obtained had a moisture content of 38.9% (W.B.). The PHA agglutinate had a Feret diameter of 25 mm.

### Comparative Example 1

An aqueous PHA suspension having a pH of 3.0 was obtained by the same method as in Example 1, except that Alcalase 2.5L (manufactured by Novozyme), which is a proteolytic enzyme, was added in the enzyme treatment such that the concentration thereof in the aqueous PHA suspension was 100 ppm. The amount of PHA surface adhesion protein was 2,500 ppm, and the liquid density was 0.95 g/mL. The volume median diameter of the PHA was 2.5 μm. When filtration was carried out by the same method as in Example 1, the filtrate permeability stood at 2,500 L/m²/hr, and the leakage ratio was 10.2%. A PHA agglutinate thus obtained had a moisture content of 40.8% (W.B.). The PHA agglutinate had a Feret diameter of 38 mm.

TABLE 1

| | | Amount of | | | | | | Moisture |
|---|---|---|---|---|---|---|---|---|
| | | PHA surface | | | | | | content of |
| | Air | adhesion | Liquid | Filtration | | Filtrate | Leakage | PHA |
| | permeability | protein | density | temperature | pH | permeability | ratio | agglutinate |
| | [cc/cm²/sec] | [ppm] | [g/mL] | [° C.] | [—] | [L/m²/hr] | [%] | [% (W.B.)] |
| Example 1 | 0.5 | 1000 | 1.00 | 60 | 3.0 | 1010 | 0.2 | 40.3 |
| Example 2 | 0.5 | 1500 | 0.95 | 60 | 3.0 | 580 | 1.0 | 38.9 |
| Comparative | 0.5 | 2500 | 0.95 | 60 | 3.0 | 2500 | 10.2 | 40.8 |
| Example 1 | | | | | | | | |

As can be seen from Table 1, when the amount of PHA surface adhesion protein is greater, the particle leakage ratio is higher.

### Example 3

An aqueous PHA suspension having a pH of 3.0 was obtained by the same method as in Example 1, except that after the pH was adjusted to 3.0 in the pH adjustment step, an aqueous PHA suspension was put in a container, and the container was shaken so that the aqueous PHA suspension was aerated. The amount of PHA surface adhesion protein was 1,000 ppm, and the liquid density was 0.7 g/mL. The volume median diameter of the PHA was 2.2 μm. When filtration was carried out by the same method as in Example 1, the filtrate permeability stood at 450 L/m²/hr, and the leakage ratio was 0.2%. A PHA agglutinate thus obtained had a moisture content of 36.8% (W.B.). The PHA agglutinate had a Feret diameter of 25 mm.

### Comparative Example 2

Filtration was carried out by the same method as in Example 1, except that a strong stir was carried out in the pH adjustment step, and the liquid density was 0.40 g/mL. The filtrate permeability stood at 150 L/m²/hr, and the leakage ratio was 3.2%. A PHA agglutinate thus obtained had a moisture content of 35.4% (W.B.). The PHA agglutinate had a Feret diameter of 34 mm.

TABLE 2

| | | Amount of | | | | | | Moisture |
|---|---|---|---|---|---|---|---|---|
| | | PHA surface | | | | | | content of |
| | Air | adhesion | Liquid | Filtration | | Filtrate | Leakage | PHA |
| | permeability | protein | density | temperature | pH | permeability | ratio | agglutinate |
| | [cc/cm²/sec] | [ppm] | [g/mL] | [° C.] | [—] | [L/m²/hr] | [%] | [% (W.B.)] |
| Example 1 | 0.5 | 1000 | 1.00 | 60 | 3.0 | 1010 | 0.2 | 40.3 |
| Example 3 | 0.5 | 1000 | 0.70 | 60 | 3.0 | 450 | 0.2 | 36.8 |
| Comparative | 0.5 | 1000 | 0.40 | 60 | 3.0 | 150 | 3.2 | 35.4 |
| Example 2 | | | | | | | | |

As can be seen from Table 2, when the liquid density is higher, the filtrate permeability becomes higher, but the moisture content is higher.

### Comparative Example 3

Filtration was carried out by the same method as in Example 1, except that the pH was adjusted to 6.0 in the pH adjustment step. The filtrate permeability stood at 11,800 L/m²/h, and the leakage ratio was 99%. No PHA agglutinate was obtained.

### Comparative Example 4

Filtration was carried out by the same method as in Example 1, except that the pH was adjusted to 2.0 in the pH adjustment step. The filtrate permeability stood at 9,800 L/m²/hr, and the leakage ratio was 95%. No PHA agglutinate was obtained.

TABLE 3

| | | Amount of | | | | | | Moisture |
|---|---|---|---|---|---|---|---|---|
| | | PHA surface | | | | | | content of |
| | Air | adhesion | Liquid | Filtration | | Filtrate | Leakage | PHA |
| | permeability | protein | density | temperature | pH | permeability | ratio | agglutinate |
| | [cc/cm²/sec] | [ppm] | [g/mL] | [° C.] | [—] | [L/m²/hr] | [%] | [% (W.B.)] |
| Example 1 | 0.5 | 1000 | 1.00 | 60 | 3.0 | 1010 | 0.2 | 40.3 |
| Comparative | 0.5 | 1000 | 1.00 | 60 | 6.0 | 11800 | 99.0 | — |
| Example 3 | | | | | | | | |
| Comparative | 0.5 | 1000 | 1.00 | 60 | 2.0 | 9800 | 95.0 | — |
| Example 4 | | | | | | | | |

As can be seen from Table 3, the leakage ratio changes due to the pH, and the pH being lower is more suitable for PHA agglutinate recovery.

### Example 4

An aqueous PHA suspension having a pH of 3.0 was obtained by the same method as in Example 1, except that a filter cloth (T7302C manufactured by Yabuta Kikai Co. Ltd.) having an air permeability of 2.0 cc/cm²/sec was used in the filtration step. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 1,210 L/m²/hr, and the leakage ratio was 0.2%. A PHA agglutinate thus obtained had a moisture content of 42.3% (W.B.). The PHA agglutinate had a Feret diameter of 17 mm.

### Comparative Example 5

Filtration was carried out by the same method as in Example 1, except that a filter cloth (PP26F manufactured by Nakao Filter Media Corp.) having an air permeability of 12.0 cc/cm²/sec was used during the filtration. The filtrate permeability stood at 9,440 L/m²/h, and the leakage ratio was 73.0%. A PHA agglutinate thus obtained had a moisture content of 47.0% (W.B.). The PHA agglutinate had a Feret diameter of 11 mm.

TABLE 4

| | | Amount of | | | | | | Moisture |
|---|---|---|---|---|---|---|---|---|
| | | PHA surface | | | | | | content of |
| | Air | adhesion | Liquid | Filtration | | Filtrate | Leakage | PHA |
| | permeability | protein | density | temperature | pH | permeability | ratio | agglutinate |
| | [cc/cm²/sec] | [ppm] | [g/mL] | [° C.] | [—] | [L/m²/hr] | [%] | [% (W.B.)] |
| Example 1 | 0.5 | 1000 | 1.00 | 60 | 3.0 | 1010 | 0.2 | 40.3 |
| Example 4 | 2.0 | 1000 | 1.00 | 60 | 3.0 | 1210 | 3.6 | 42.3 |
| Comparative | 12.0 | 1000 | 1.00 | 60 | 3.0 | 9440 | 73.0 | 47.0 |
| Example 5 | | | | | | | | |

As can be seen from Table 4, when a filter medium having a high air permeability is used, the filtrate permeability increases, but the leakage ratio increases as well.

### Example 5

An aqueous PHA suspension having a pH of 3.0 was obtained by the same method as in Example 1, except that heating was carried out in the filtration step until the temperature of the aqueous PHA suspension became 75° C. in a water bath having a temperature of 78° C. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 1,580 L/m²/hr, and the leakage ratio was 0.3%. A PHA agglutinate thus obtained had a moisture content of 42.5% (W.B.). The PHA agglutinate had a Feret diameter of 18 mm.

### Example 6

An aqueous PHA suspension having a pH of 3.0 was obtained by the same method as in Example 1, except that cooling was carried out in the filtration step until the temperature of the aqueous PHA suspension became 25° C. in a water bath having a temperature of 28° C. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 450 L/m²/hr, and the leakage ratio was 0.1%. A PHA agglutinate thus obtained had a moisture content of 44.8% (W.B.). The PHA agglutinate had a Feret diameter of 26 mm.

TABLE 5

| | | Amount of | | | | | | Moisture |
|---|---|---|---|---|---|---|---|---|
| | | PHA surface | | | | | | content of |
| | Air | adhesion | Liquid | Filtration | | Filtrate | Leakage | PHA |
| | permeability | protein | density | temperature | pH | permeability | ratio | agglutinate |
| | [cc/cm²/sec] | [ppm] | [g/mL] | [° C.] | [—] | [L/m²/hr] | [%] | [% (W.B.)] |
| Example 1 | 0.5 | 1000 | 1.00 | 60 | 3.0 | 1010 | 0.2 | 40.3 |
| Example 5 | 0.5 | 1000 | 1.00 | 75 | 3.0 | 1580 | 0.3 | 42.5 |
| Example 6 | 0.5 | 1000 | 1.00 | 25 | 3.0 | 450 | 0.1 | 44.8 |

As can be seen from Table 5, the temperature of the aqueous PHA suspension affects the filtrate permeability. When the temperature was higher, the filtrate permeability became higher.

### Example 7

An aqueous PHA suspension having a pH of 3.0 was obtained by the same method as in Example 1, except that before the filtration step, a liquid after the pH adjustment step was heated until the temperature of the liquid became 75° C. in an oil bath having a temperature of 125° C., and then cooled until the temperature of the liquid became 60° C. in a water bath having a temperature of 63° C. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 1,220 L/m²/hr, and the leakage ratio was 0.2%. A PHA agglutinate thus obtained had a moisture content of 42.2% (W.B.). The PHA agglutinate had a Feret diameter of 28 mm.

### Example 8

An aqueous PHA suspension having a pH of 3.0 was obtained by the same method as in Example 1, except that before the filtration step, a liquid after the pH adjustment step was heated until the temperature of the liquid became 90° C. in an oil bath having a temperature of 125° C., and then cooled until the temperature of the liquid became 60° C. in a water bath having a temperature of 63° C. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 1,320 L/m²/hr, and the leakage ratio was 0.1%. A PHA agglutinate thus obtained had a moisture content of 44.0% (W.B.). The PHA agglutinate had a Feret diameter of 21 mm.

### Example 9

An aqueous PHA suspension having a pH of 3.0 was obtained by the same method as in Example 1, except that: the solid concentration of the aqueous suspension liquid was adjusted to 28% by weight in the pH adjustment step; a liquid after the pH adjustment step was put in a pressure-resistant pot before the filtration step, steam having a pressure of 0.3 MPaG was fed, the liquid was heated until the temperature thereof became 110° C., and then cooled until the temperature thereof became 60° C. in a water bath having a temperature of 63° C.; and distillated water was added so that the solid concentration of the aqueous PHA suspension became 25% by weight. The amount of PHA surface adhesion protein was 1,000 ppm, and the liquid density was 1.00 g/mL. The volume median diameter of the PHA was 2.2 μm. When filtration was carried out by the same method as in Example 1, the filtrate permeability stood at 2,840 L/m²/hr, and the leakage ratio was 0.0%. A PHA agglutinate thus obtained had a moisture content of 44.9% (W.B.). The PHA agglutinate had a Feret diameter of 15 mm.

TABLE 6

| | | Amount of | | | | | | | Moisture |
|---|---|---|---|---|---|---|---|---|---|
| | | PHA surface | | Heat | | | | | content of |
| | Air | adhesion | Liquid | treatment | Filtration | | Filtrate | Leakage | PHA |
| | permeability | protein | density | temperature | temperature | pH | permeability | ratio | agglutinate |
| | [cc/cm²/sec] | [ppm] | [g/mL] | [° C.] | [° C.] | [—] | [L/m²/hr] | [%] | [% (W.B.)] |
| Example 1 | 0.5 | 1000 | 1.00 | none | 60 | 3.0 | 1010 | 0.2 | 40.3 |
| Example 7 | 0.5 | 1000 | 1.00 | 75 | 60 | 3.0 | 1220 | 0.2 | 42.2 |
| Example 8 | 0.5 | 1000 | 1.00 | 90 | 60 | 3.0 | 1320 | 0.1 | 44.0 |
| Example 9 | 0.5 | 1000 | 1.00 | 110 | 60 | 3.0 | 2840 | 0.0 | 44.9 |

As can be seen from Table 6, once the aqueous PHA suspension is heated to high temperature, the filtrate permeability increases, and when the heating temperature is higher, the filtrate permeability increases to a higher degree.

### Example 10

The same operations as in Example 1 were carried out before and in the pH adjustment step. Pressure filtration was carried out via a press filter (YTO filter press manufactured by Yabuta Kikai Co. Ltd.) with use of a filter cloth (T7104C manufactured by Yabuta Kikai Co. Ltd.) having an air permeability of 0.5 cc/cm²/sec). When calculated by the above described method, the filtrate permeability was 1,500 L/m²/hr. Further, when press dewatering was carried out under a pressure of 0.5 MPa, a PHA agglutinate thus obtained had a moisture content of 31.9% (W.B.). The leakage ratio of the whole filtrate thus obtained was 2.6%. Regardless of the filtration method, filtration was successfully carried out. The PHA agglutinate had a Feret diameter of 20 mm.

### Example 11

### (Drying)

The PHA agglutinate obtained in Example 1 was placed in a dryer (WFO-700 manufactured by EYELA) and dried at 60° C. for 24 hours. The PHA agglutinate dried was redispersed in water, and the solid concentration was adjusted to 15% by weight. The pH was adjusted between 7 to 9 with use of 1% NaOH aqueous solution and 1% H₂SO₄ aqueous solution, and the PHA agglutinate redispersed in water was stirred. An aqueous PHA suspension was thus prepared. When the particle size of PHA particles in the aqueous PHA suspension was measured after 30 minutes of stirring, the volume median diameter was 2.8 μm.

### CONCLUSION

In view of the above, it has been found that according to the present production method, the amount of PHA surface adhesion protein, a liquid density, the pH of an aqueous suspension liquid, the air permeability of a filter medium, and the temperature of the aqueous PHA suspension affects the filtrate permeability and the leakage ratio during filtration. It has thus been found that by controlling at least the amount of PHA surface adhesion protein, liquid density, pH of an aqueous PHA suspension, and air permeability of a filter medium, it is possible to efficiently obtain a PHA by filtration. In addition, it has been revealed that with the present production method, it is possible to obtain a PHA by filtration regardless of the filtration method.

### INDUSTRIAL APPLICABILITY

With the present production method, it is possible to produce PHAs through an easy and simple operation. This makes it possible to advantageously use the present production method in production of PHAs. Furthermore, PHAs obtained by the present production method can be suitably used in the fields of agriculture, fishery, forestry, horticulture, medicine, hygiene, clothing, non-clothing, packaging, motor vehicles, and building materials, and any other fields.

1. A method of polyhydroxyalkanoate production, the method comprising:
subjecting an aqueous polyhydroxyalkanoate suspension having a pH of 2.5 to 5.5 to dead-end filtration using a filter medium having an air permeability of from 0.01 cc/cm²/sec to 5.0 cc/cm²/sec,
wherein the aqueous polyhydroxyalkanoate suspension comprises a polyhydroxyalkanoate surface adhesion protein in an amount of not more than 2,000 ppm, and
the aqueous polyhydroxyalkanoate suspension has a liquid density of from 0.50 g/mL to 1.08 g/mL in the filtration.

2. The method according to claim 1, wherein in the filtration, a filtrate permeability is at least 200 L/m²/hr, and a leakage ratio is not more than 5%.

3. The method according to claim 1, wherein the aqueous polyhydroxyalkanoate suspension has a temperature of from 20° C. to 95° C. in the filtration.

4. The method according to claim 1, further comprising (a) disrupting and solubilizing cell-derived components of a microbial cell comprising a polyhydroxyalkanoate, wherein the cell-derived components exclude the polyhydroxyalkanoate, and
the polyhydroxyalkanoate has a volume median diameter of from 0.5 μm to 5.0 μm in the (a).

5. The method according to claim 4, further comprising (b) recovering the aqueous polyhydroxyalkanoate suspension by centrifugation after the (a).

6. The method according to claim 1, further comprising drying a polyhydroxyalkanoate obtained in the filtration at 20° C. to 80° C., thereby obtaining a dried polyhydroxyalkanoate.

7. The method according to claim 6, further comprising redispersing, in an aqueous solvent, the dried polyhydroxyalkanoate, to obtain an aqueous polyhydroxyalkanoate suspension which comprises a polyhydroxyalkanoate having a volume median diameter of from 0.5 μm to 5.0 μm.

8. The method according to claim 1, further comprising subjecting the aqueous polyhydroxyalkanoate suspension to a heat treatment before the filtration such that the aqueous polyhydroxyalkanoate suspension has a temperature of from 60° C. to 120° C.

9. The method according to claim 8, wherein the aqueous polyhydroxyalkanoate suspension has a temperature during the filtration at least 5° C. lower than after the heat treatment.

10. The method according to claim 5, further comprising adjusting a pH of the aqueous polyhydroxyalkanoate suspension obtained in the (b) to from 2.5 to 5.5.

11. (canceled)

12. The method of claim 4, wherein the polyhydroxyalkanoate has the volume median diameter of from 1.0 μm to 4.5 μm in the (a).

13. The method of claim 1, wherein the air permeability of the filter medium is from 0.1 cc/cm²/sec to 4.0 cc/cm²/sec.

14. The method of claim 1, wherein the liquid density of the aqueous polyhydroxyalkanoate suspension is from 0.60 g/mL to 1.05 g/mL.

15. The method of claim 2, wherein the filtrate permeability is at least 300 L/m²/hr, and a leakage ratio is not more than 3%.

16. The method of claim 3, wherein the aqueous polyhydroxyalkanoate suspension has the temperature of from 20° C. to 90° C. in the filtration.

17. The method according to claim 1, wherein in the filtration, a filtrate permeability is at least 200 L/m²/hr.

18. The method according to claim 1, wherein in the filtration, a leakage ratio is not more than 5%.

19. The method according to claim 1,
wherein the aqueous polyhydroxyalkanoate suspension has a temperature of from 20° C. to 95° C. in the filtration,
the method further comprising:
(a) disrupting and solubilizing cell-derived components of a microbial cell comprising a polyhydroxyalkanoate, wherein the cell-derived components exclude the polyhydroxyalkanoate, and the polyhydroxyalkanoate has a volume median diameter of from 0.5 μm to 5.0 μm in the (a),
(b) recovering the aqueous polyhydroxyalkanoate suspension by centrifugation after the (a), and
adjusting a pH of the aqueous polyhydroxyalkanoate suspension obtained in the (b) to from 2.5 to 5.5.

20. The method according to claim 1,
wherein the aqueous polyhydroxyalkanoate suspension has a temperature of from 20° C. to 95° C. in the filtration,
the method further comprising:
subjecting the aqueous polyhydroxyalkanoate suspension to a heat treatment before the filtration such that the aqueous polyhydroxyalkanoate suspension has a temperature of from 60° C. to 120° C.
